SEQUENCE LISTING (14 sequences)

SEQ ID NO: 1 - DNA, 18 bp, Artificial Sequence
  ayg1F
cactgcacgtaatcaaga

SEQ ID NO: 2 - DNA, 19 bp, Artificial Sequence
  ayg1R
cctgagtgtattcatccga

SEQ ID NO: 3 - DNA, 21 bp, Artificial Sequence
  ayg1-LC 640
tgagcccatcttcaaccatct

SEQ ID NO: 4 - DNA, 19 bp, Artificial Sequence
  ayg1-flu
gacggctccatcgaggact

SEQ ID NO: 5 - DNA, 301 bp, Aspergillus fumigatus
tgccacgctggatccttggagacaagttcgataccgtcttcccgcacaagggctcgttgaaggtcctgtgggagtcgagatggaagtttgctgtatgtctttcccctagaaactagaggccctctaatagtcgtcagtgttcaaaatccgtctatcccttccacgacggctccatcgaggactttgagcccatcttcaaccatctcatatcggtatgctcttgatcttcacgacacgacagccagcctgctgacccgacagaaaaacatcaacgacgccgcctcggatgaatacactcagg

SEQ ID NO: 6 - DNA, 301 bp, Aspergillus fumigatus; identical to SEQ ID NO: 5

SEQ ID NO: 7 - DNA, 301 bp, Aspergillus fumigatus; identical to SEQ ID NO: 5

SEQ ID NO: 8 - DNA, 301 bp, Aspergillus fumigatus; identical to SEQ ID NO: 5

SEQ ID NO: 9 - DNA, 301 bp, Aspergillus fumigatus; identical to SEQ ID NO: 5

SEQ ID NO: 10 - DNA, 301 bp, Aspergillus fumigatus; identical to SEQ ID NO: 5

SEQ ID NO: 11 - DNA, 301 bp, Aspergillus fumigatus
tgccacgatggatccttggagacaagttcgataccgtcttcccgcacaagggctcgttgaaggtcctgtgggagtcgagatggaagtttgctgtatgtctttcccctagaaactagaggccctctaatagtcgtcagtgttcaaaatccgtctatcccttccacgacggctccatcgaggactttgagcccatcttcaaccatctcatatcggtatgctcttgatcttcacgacacgacagccagcctgctgacccgacagaaaaacatcaatgacgccgcctcggatgaatacactcagg

SEQ ID NO: 12 - DNA, 301 bp, Aspergillus fumigatus; identical to SEQ ID NO: 5

SEQ ID NO: 13 - DNA, 301 bp, Aspergillus fumigatus; identical to SEQ ID NO: 5

SEQ ID NO: 14 - DNA, 301 bp, Aspergillus fumigatus; identical to SEQ ID NO: 11

(19) United States
(12) Patent Application Publication
(10) Pub. No.: US 2011/0311969 A1
(43) Pub. Date: Dec. 22, 2011

(54) Diagnostic Kit for Aspergillus Fumigatus Species

(75) Inventors: Louise O'Connor, Co. Galway (IE); Majella Maher, Galway (IE)

(73) Assignee: NATIONAL UNIVERSITY OF IRELAND, GALWAY, Galway (IE)

(21) Appl. No.: 13/060,633

(22) PCT Filed: Jul. 10, 2009
(86) PCT No.: PCT/EP2009/058858

§ 371 Date: Jun. 9, 2011

(30) Foreign Application Priority Data
Jul. 10, 2008 (IE) 2008/0567

(51) Int. Cl. C12Q 1/68; C07H 21/02; C07H 21/04

(52) U.S. Cl. 435/6.11; 536/24.32

(57) ABSTRACT

The use of the Ayg1 gene or an RNA transcript of the Ayg1 gene or fragments thereof as target regions in a diagnostic assay for the eukaryotic organism Aspergillus fumigatus species is described. The unique sequence of the Ayg1 gene in Aspergillus fumigatus provides the basis of nucleic acid diagnostic test for the identification of the pathogen at a the molecular level.

### FIELD OF THE INVENTION

The present invention relates to nucleic acid primers and probes for use in the detection and identification of Aspergillus fumigatus species. More specifically, the present invention relates to the Ayg1 gene, the corresponding RNA, specific probes, primers and oligonucleotides related thereto, and their use in an assay kit to detect Aspergillus fumigatus species.

### BACKGROUND TO THE INVENTION

Hospital acquired infections result in over 80,000 deaths a year in the US alone and subsequently ensue a significant cost burden on the hospital. Aspergillus fumigatus causes more hospital-acquired infections worldwide than any other fungus. Aspergillus fumigatus is ubiquitous in the environment and is particularly common in soil and decaying vegetation, where it survives on organic debris. The main portal of entry in the human body and site of infection is the respiratory tract.

Recent statistics report that 4% of all patients dying in tertiary care hospitals in Europe have invasive aspergillosis (IA). Four types of IA have been described, acute or chronic pulmonary aspergillosis, which is the most common form of IA, tracheobronchitis and obstructive bronchial disease, acute invasive rhinosinusitis and lastly, disseminated disease commonly involving the brain. The clinical manifestations and severity of the disease depends on the immunologic state of the patient. Low resistance in patients is due to factors such as, underlying debilitating disease, neutropenia chemotherapy, disruption of normal flora and an inflammatory response due to the use of antimicrobial agents and steroids. These factors can predispose patients to colonisation, invasive disease or both. Patients particularly at risk include lung, heart, renal and bone marrow transplant recipients, leukaemia and AIDS patients. The main symptoms of IA are similar to those of other forms of aspergillus infection and are generally non-specific and variable. The symptoms include fever, chest pain, malaise and weight loss.

Many advances have been made in the treatment of IA in recent years. Voriconazle, amphotericin B and itraconazole are the anti-fungal agents most effective in the treatment of patient suffering from IA. Despite such therapeutic advancements, IA is nonetheless associated with significant morbidity and mortality.

Current laboratory diagnostic methods for IA involve serological detection of circulating fungal antigens or immunoprecipitation. Such methods are, however, time consuming and do not always yield accurate results and detection. Diagnostic methods centred upon histological analysis of biopsies are highly sensitive and specific but are frequently associated with bleeding complications. Difficulties with diagnosis mean that most invasive fungal infections are proven only at autopsy. The lack of early detection of Aspergillus fumigatus in blood and BAL/sputum samples is not only a significant problem for management of patient health but is also resulting in increased prophylactic antifungal use which in turn is accelerating development of drug resistance. Due to the difficulty in the diagnosis of Aspergillus fumigatus and the rapid progression of the disease, a large number of patients are treated prior to the confirmation of the diagnosis.

Therefore, in order to overcome the limitations of the traditional culture-based and serological based detection methods, efforts are ongoing to develop a less invasive, reliable and specific diagnostic test for IA, capable of detecting even non-viable cells or circulating fungal DNA. Currently described molecular detection methods for A. fumigatus are based largely on ribosomal 18S, 28S, 5.8S rRNA genes and/or ITS1 and ITS2 intergenic spacer sequences. Intergenic spacer sequences are non-coding sequences that are not associated with virulence of the pathogen. Detection methods for the specific identification of Aspergillus species in clinical samples, such as real-time PCR, DNA microarrays, panfungal PCR amplification in combination with DNA sequencing methods, utilising the above mentioned targets, have been recently described in the literature (De Marco et al., 2007; Speiss et al., 2007; Lan et al., 2007). A study undertaken by Balajee et al., 2007, highlights the limitations of relying on phenotypic methods for the identification of clinically relevant fungal species, recommending a combination of ITS1 and β tubulin gene sequencing to improve identification to species level. There are several commercially available molecular tests for Aspergillus species identification, including the Applied Biosystems MicroSeq D2 system, which can be used to identify Aspergillus species based on the sequence of the D2 region of the large-submit ribosomal RNA gene (LSU). The SeptiFast Kit specifically identifies A. fumigatus by real-time PCR targeting the ITS1 intergenic spacer sequence. Ribosomal genes and ITS1-2 sequences are universally present in all yeast and fungal species. This has a disadvantage in that if one wants to identify a fungal pathogen in a test sample in which Candida is also present, there is a possibility of competition between the Candida and the fungal species in the amplification process and the Candida species may be preferably amplified, potentially resulting in a false-positive and a failure to detect the pathogen causing the disease in question.

WO20010066790 discloses a method of detecting Aspergillus fumigatus based on the specific enzymatic activity present in the microorganism, in particular the arabinopyranoside substrate cleavage activity. This method however, is laborious and time-consuming WO2002079512 and WO1996021741 describe a method for detecting Aspergillus fumigatus by means of amplifying the 18S ribosomal target gene and the 28S ribosomal target gene, respectively. In addition, neither the 18S gene nor the 28S gene are associated with virulence and thus, are not specifically associated with infection.

Aspergillus fumigatus conidia produce a bluish-green pigment through a pentaketide pathway. The pigment contains a 1,8-dihydroxnaphthalene (DHN)-like pentakedide melanin and plays a major role in protection of the fungus, against factors such as immune effector cells. Genetic and biochemical investigations have shown that biosynthesis of the pentaketide melanin requires a developmentally regulated six-gene cluster that includes the genes alb1, arp2, arp1, yA and ayg1, Alb1, arp2 and arp1 encode PKS, 1,3,6,9-THN reductase and scytalone dehydratase, respectively. A study by Huei-Fung Tsai et al., 1999 reported that disruption of the ayg1 gene prevented accumulation of 1,3,6,9-THN reductase.

It is clear that early and specific diagnosis of infection is vital. The severity and rapid progression of infection with A. fumigatus, in immunocomprised patients in particular, illustrates the need for a new effective and sensitive diagnostic method. The current inventors have shown the potential ayg1 gene regions for use in the molecular identification of the fungal species A. fumigatus. Although, the agy1 gene may be present in other Aspergillus species, the gene sequence of the ayg1 gene in A. fumigatus is unique and as such, is an ideal candidate for the development of a rapid, accurate, specific diagnostic test for A. fumigatus, thereby resolving the deficiencies of previously employed techniques.

### DEFINITIONS

“Synthetic oligonucleotide” refers to molecules of nucleic acid polymers of 2 or more nucleotide bases that are not derived directly from genomic DNA or live organisms. The term synthetic oligonucleotide is intended to encompass DNA, RNA, and DNA/RNA hybrid molecules that have been manufactured chemically, or synthesized enzymatically in vitro.

An “oligonucleotide” is a nucleotide polymer having two or more nucleotide subunits covalently joined together. Oligonucleotides are generally about 10 to about 100 nucleotides. The sugar groups of the nucleotide subunits may be ribose, deoxyribose, or modified derivatives thereof such as OMe. The nucleotide subunits may be joined by linkages such as phosphodiester linkages, modified linkages or by non-nucleotide moieties that do not prevent hybridization of the oligonucleotide to its complementary target nucleotide sequence. Modified linkages include those in which a standard phosphodiester linkage is replaced with a different linkage, such as a phosphorothioate linkage, a methylphosphonate linkage, or a neutral peptide linkage. Nitrogenous base analogs also may be components of oligonucleotides in accordance with the invention.

A “target nucleic acid” is a nucleic acid comprising a target nucleic acid sequence. A “target nucleic acid sequence,” “target nucleotide sequence” or “target sequence” is a specific deoxyribonucleotide or ribonucleotide sequence that can be hybridized to a complementary oligonucleotide.

An “oligonucleotide probe” is an oligonucleotide having a nucleotide sequence sufficiently complementary to its target nucleic acid sequence to be able to form a detectable hybrid probe:target duplex under high stringency hybridization conditions. An oligonucleotide probe is an isolated chemical species and may include additional nucleotides outside of the targeted region as long as such nucleotides do not prevent hybridization under high stringency hybridization conditions. Non-complementary sequences, such as promoter sequences, restriction endonuclease recognition sites, or sequences that confer a desired secondary or tertiary structure such as a catalytic active site can be used to facilitate detection using the invented probes. An oligonucleotide probe optionally may be labelled with a detectable moiety such as a radioisotope, a fluorescent moiety, a chemiluminescent, a nanoparticle moiety, an enzyme or a ligand, which can be used to detect or confirm probe hybridization to its target sequence. Oligonucleotide probes are preferred to be in the size range of from about 10 to about 100 nucleotides in length, although it is possible for probes to be as much as and above about 500 nucleotides in length, or below 10 nucleotides in length.

A “hybrid” or a “duplex” is a complex formed between two single-stranded nucleic acid sequences by Watson-Crick base pairings or non-canonical base pairings between the complementary bases. “Hybridization” is the process by which two complementary strands of nucleic acid combine to form a double-stranded structure (“hybrid” or “duplex”). A “fungus” or “yeast” is meant any organism of the kingdom Fungi, and preferably, is directed towards any organism of the phylum Ascomycota.

“Complementarity” is a property conferred by the base sequence of a single strand of DNA or RNA which may form a hybrid or double-stranded DNA:DNA, RNA:RNA or DNA:RNA through hydrogen bonding between Watson-Crick base pairs on the respective strands. Adenine (A) ordinarily complements thymine (T) or uracil (U), while guanine (G) ordinarily complements cytosine (C).

The term “stringency” is used to describe the temperature, ionic strength and solvent composition existing during hybridization and the subsequent processing steps. Those skilled in the art will recognize that “stringency” conditions may be altered by varying those parameters either individually or together. Under high stringency conditions only highly complementary nucleic acid hybrids will form; hybrids without a sufficient degree of complementarity will not form. Accordingly, the stringency of the assay conditions determines the amount of complementarity needed between two nucleic acid strands forming a hybrid. Stringency conditions are chosen to maximize the difference in stability between the hybrid formed with the target and the non-target nucleic acid.

With “high stringency” conditions, nucleic acid base pairing will occur only between nucleic acid fragments that have a high frequency of complementary base sequences (for example, hybridization under “high stringency” conditions, may occur between homologs with about 85-100% identity, preferably about 70-100% identity). With medium stringency conditions, nucleic acid base pairing will occur between nucleic acids with an intermediate frequency of complementary base sequences (for example, hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

‘High stringency’ conditions are those equivalent to binding or hybridization at 42° C. in a solution consisting of 5×SSPE (43.8 g/l NaCl, 6.9 g/l NaH₂PO₄H₂O and 1.85 g/l EDTA, ph adjusted to 7.4 with NaOH), 0.5% SDS, 5×Denhardt's reagent and 100 m/ml denatured salmon sperm DNA followed by washing in a solution comprising 0.1×SSPE, 1.0% SDS at 42° C. when a probe of about 500 nucleotides in length is used.

“Medium stringency’ conditions are those equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C., when a probe of about 500 nucleotides in length is used.

‘Low stringency’ conditions are those equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C., when a probe of about 500 nucleotides in length is used.

In the context of nucleic acid in-vitro amplification based technologies, “stringency” is achieved by applying temperature conditions and ionic buffer conditions that are particular to that in-vitro amplification technology. For example, in the context of PCR and real-time PCR, “stringency” is achieved by applying specific temperatures and ionic buffer strength for hybridisation of the oligonucleotide primers and, with regards to real-time PCR hybridisation of the probe/s, to the target nucleic acid for in-vitro amplification of the target nucleic acid.

One skilled in the art will understand that substantially corresponding probes of the invention can vary from the referred-to sequence and still hybridize to the same target nucleic acid sequence. This variation from the nucleic acid may be stated in terms of a percentage of identical bases within the sequence or the percentage of perfectly complementary bases between the probe and its target sequence. Probes of the present invention substantially correspond to a nucleic acid sequence if these percentages are from about 100% to about 80% or from 0 base mismatches in about 10 nucleotide target sequence to about 2 bases mismatched in an about 10 nucleotide target sequence. In preferred embodiments, the percentage is from about 100% to about 85%. In more preferred embodiments, this percentage is from about 90% to about 100%; in other preferred embodiments, this percentage is from about 95% to about 100% e.g., 95, 96, 97, 98, 99, or 100%.

The terms “identical” or percent “identity,” in the context of two or more nucleic acids or polypeptide sequences, refer to two or more sequences or subsequences that are the same or have a specified percentage of amino acid residues or nucleotides that are the same (i.e., 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site at ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

By “sufficiently complementary” or “substantially complementary” is meant nucleic acids having a sufficient amount of contiguous complementary nucleotides to form, under high stringency hybridization conditions, a hybrid that is stable for detection.

By “nucleic acid hybrid” or “oligonucleotide:target duplex” is meant a structure that is a double-stranded, hydrogen-bonded structure, preferably about 10 to about 100 nucleotides in length, more preferably 14 to 50 nucleotides in length, although this will depend to an extent on the overall length of the oligonucleotide probe. The structure is sufficiently stable to be detected by means such as chemiluminescent or fluorescent light detection, autoradiography, electrochemical analysis or gel electrophoresis. Such hybrids include RNA:RNA, RNA:DNA, or DNA:DNA duplex molecules.

“RNA and DNA equivalents” refer to RNA and DNA molecules having the same complementary base pair hybridization properties. RNA and DNA equivalents have different sugar groups (i.e., ribose versus deoxyribose), and may differ by the presence of uracil in RNA and thymine in DNA. The difference between RNA and DNA equivalents do not contribute to differences in substantially corresponding nucleic acid sequences because the equivalents have the same degree of complementarity to a particular sequence.

By “preferentially hybridize” is meant that under high stringency hybridization conditions oligonucleotide probes can hybridize their target nucleic acids to form stable probe:target hybrids (thereby indicating the presence of the target nucleic acids) without forming stable probe:non-target hybrids (that would indicate the presence of non-target nucleic acids from other organisms). Thus, the probe hybridizes to target nucleic acid to a sufficiently greater extent than to non-target nucleic acid to enable one skilled in the art to accurately detect the presence of (for example Candida) and distinguish these species from other organisms. Preferential hybridization can be measured using techniques known in the art and described herein.

By “theranostics” is meant the use of diagnostic testing to diagnose the disease, choose the correct treatment regime and monitor the patient response to therapy. The theranostics of the invention may be based on the use of an NAD assay of this invention on samples, swabs or specimens collected from the patient.

### OBJECT OF THE INVENTION

It is an object of the current invention to provide sequences and/or diagnostic kits to detect and identify A. fumigatus fungal species. The current inventors have made use of the ayg1 gene sequence to design primers and probes that are specific to A. fumigatus.

### SUMMARY OF THE INVENTION

The present invention provides a diagnostic kit for detection and identification of Aspergillus fumigatus, comprising at least one oligonucleotide probe sequence capable of hybridising to at least a portion of the ayg1 gene or its corresponding mRNA. The probe is capable of hybridising with a complementary DNA or RNA molecule. The probe can have a sequence of SEQ ID NO 3 or SEQ ID NO 4, or a sequence substantially homologous to or substantially complementary to those sequences, which can also act as a probe for the ayg1 gene. The portion of the ayg 1 gene may be equivalent to a portion of the region of the gene from base pair position 316 to base pair 634 of the gene. The kit may comprise a plurality of probes. In addition the kit may comprise additional probes for other organisms, such as, for example, bacterial species or viruses. The oligonucleotide probe may be synthetic.

The kit may further comprise a primer for amplification of at least a portion of the ayg1 gene. Suitably, the kit comprises at least one forward and at least one reverse in vitro amplification primer for a portion of the ayg1 gene. The primer may have a sequence selected from the group comprising SEQ ID NO 1 or SEQ ID NO 2 or a sequence substantially homologous to or substantially complementary to those sequences, which can also act as a primer for the ayg1 gene. The portion of the ayg 1 gene may be equivalent to a portion of the region of the gene corresponding to base pair positions 316 to base pair 634 of the gene.

A kit for detecting or identifying a Aspergillus fumigatus ayg1 polynucleotide comprises an oligonucleotide probe which preferentially hybridizes to the same nucleotide sequence as is preferentially hybridized by SEQ ID NO: 3 or 4 and further comprises a forward primer which preferentially hybridizes to the same nucleotide sequence as is preferentially hybridized by SEQ ID NO: 1 and/or a reverse primer which preferentially hybridizes to the same nucleotide sequence as is preferentially hybridized by SEQ ID NO: 2.

The identified sequences are suitable not only for in vitro DNA/RNA amplification based detection systems but also for signal amplification based detection systems. The detection system may be based on direct nucleic acid detection technologies, signal amplification nucleic acid detection technologies, and nucleic acid in vitro amplification technologies selected from one or more of Polymerase Chain Reaction (PCR), Ligase Chain Reaction (LCR), Nucleic Acids Sequence Based Amplification (NASBA), Strand Displacement Amplification (SDA), Transcription Mediated Amplification (TMA), Branched DNA technology (bDNA) and Rolling Circle Amplification Technology (RCAT), or other in vitro enzymatic amplification technologies.

The current invention further provides an oligonucleotide sequence selected from the group consisting of: SEQ ID NO 1 through SEQ ID NO 14 and sequences substantially homologous or substantially complementary thereto or to a portion thereof and having a function in diagnostics based on the ayg1 gene. The oligonucleotide sequence may comprise DNA. The oligonucleotide may comprise RNA. The oligonucleotide may comprise a mixture of DNA, RNA and PNA. The oligonucleotide may comprise synthetic nucleotides.

The oligonucleotides of the invention may be provided in a composition for detection. Such a composition may comprise buffers, enzymes, detergents, salts and so on, as appropriate to the intended use of the compositions. It is also envisioned that the compositions, kits and methods of the invention, while described herein as comprising at least one synthetic oligonucleotide, may also comprise natural oligonucleotides with substantially the same sequences as the synthetic nucleotide fragments in place of, or alongside synthetic oligonucleotides

The oligonucleotide sequences and kits of the present invention may be used in a detection assay to detect the presence of A. fumigatus, to detect A. fumigatus in a patient, to measure A. fumigatus titres in a patient or a methods of assessing the efficacy and response of a treatment regime designed to reduce A. fumigatus species titre in a patient. In one embodiment, the kit can be used for the identification of A. fumigatus in an environmental sample, in a food sample, in an industrial sample or a clinical sample. The environment may be a hospital, or it may be a food sample, an environmental sample, e.g. water, an industrial sample such as an in-process sample or an end product requiring bioburden or quality assessment. Ideally, the test sample is a lysate of an obtained sample from a patient (such as a swab, or blood, urine, saliva, a bronchial lavage dental specimen, skin specimen, scalp specimen, transplant organ biopsy, stool, mucus, or discharge sample). The invention further provides a method of assessing the efficacy of a treatment regime designed to reduce fungal titre in a patient comprising the use of the kits, oligonucleotide sequences or methods of the current invention at one or more key stages of the treatment regime.

The invention also provides kits for use in theranostics, food safety diagnostics, industrial microbiology diagnostics, environmental monitoring, veterinary diagnostics, bio-terrorism diagnostics comprising one or more of the synthetic oligonucleotides of the invention. The kits may also comprise one or more articles selected from the group consisting of appropriate sample collecting instruments, reagent containers, buffers, labelling moieties, solutions, detergents and supplementary solutions. The invention also provides for use of the sequences, compositions, nucleotide fragments, assays, and kits of the invention in theranostics, Food safety diagnostics, Industrial microbiology diagnostics, Environmental monitoring, Veterinary diagnostics, Bio-terrorism diagnostics.

The oligonucleotides, kits or methods may be used in a diagnostic nucleic acid based assay for the detection of A. fumigatus. The oligonucleotide, kits or methods may be used in a diagnostic assay to measure fungal titres in a patient. The titres may be measured in vitro.

The kits and the nucleic acid molecule of the invention may be used in the identification and/or characterization of one or more disruptive agents that can be used to disrupt the ayg1 gene function. The disruptive agent may be selected from the group consisting of antisense RNA, PNA, mirRNA and siRNA.

The invention also provides a diagnostic kit for a target fungal organism comprising an oligonucleotide probe substantially homologous to or substantially complementary to an oligonucleotide of the invention, which may be synthetic. It will be appreciated that sequences suitable for use as in vitro amplification primers may also be suitable for use as oligonucleotide probes: while it is preferable that amplification primers may have a complementary portion of between about 15 nucleotides and about 30 nucleotides (more preferably about 15-23), oligonucleotides probes of the invention may be any suitable length. The skilled person will appreciate that different hybridisation and or annealing conditions will be required depending on the length, nature and structure (e.g. hybridisation probe pairs for LightCycler, Taqman 5′ exonuclease probes, hairpin loop structures etc, and sequence of the oligonucletide probe selected). Kits and assays of the invention may also be provided wherein the oligonucleotide probe is immobilized on a surface. Such a surface may be a bead, a membrane, a column, dipstick, a nanoparticle, the interior surface of a reaction chamber such as a well of a diagnostic plate or inside of a reaction tube, capillary or vessel or the like.

The current invention further provides a method of detecting of A. fumigatus in a test sample comprising the steps of:
(i) Mixing the test sample with at least one oligonucleotide sequence capable of binding to at least a portion of the ayg1 gene or its corresponding mRNA under appropriate conditions;
(ii) Hybridising under defined conditions any nucleic acid that may be present in the test sample with the oligonucleotide sequence to form a sequence:target duplex; and
(iii) Determining whether an sequence:target duplex is present; the presence of the duplex positively identifying the presence of the target organism in the test sample.

The oligonucleotide sequence may be selected from the group consisting of SEQ ID No1 to SEQ ID No 4 or sequences substantially homologous or substantially complementary thereto also capable of acting as a probe or primer for the ayg1 gene. The sample may be an environmental sample, an industrial sample, a clinical sample or a food sample. The environment may be a hospital or it may be a food sample, an environmental sample e.g. water, an industrial sample such as an in-process sample or an end product requiring bioburden or quality assessment. The test sample may comprise cells of the target organism. The method may also comprise a step for releasing nucleic acid from any cells of the target organism that may be present in said test sample. Ideally, the test sample is a lysate of an obtained sample from a patient (such as a swab, or blood, urine, saliva, a bronchial lavage dental specimen, skin specimen, scalp specimen, transplant organ biopsy, stool, mucus, or discharge sample).

The current invention will now be described with reference to the following examples and figures. It is to be understood that the following detailed description and accompanying figures, are exemplary and explanatory only and are intended to provide a further explanation of the present invention, as claimed and not to limit the scope of the invention in any way.

### BRIEF DESCRIPTION OF THE DRAWINGS

FIG. 1: Alignment of partial ayg1 sequences for A. fumigatus strains

FIG. 2: Amplification curves obtained for A. fumigatus strains tested using the ayg1 detection assay. All A. fumigatus strains (n=12) tested were detected in the assay.

FIG. 3: Specificity of the A. fumigatus ayg1 assay testing a range of related Aspergillus species. No cross-reaction was observed.

FIG. 4: Specificity of the A. fumigatus ayg1 assay testing a range of bacterial species and Human DNA. No cross-reaction was observed.

FIG. 5 (a) Detection limit of the ayg1 assay for A. fumigatus. Amplification of the ayg1 gene target from serially diluted A. fumigatus DNA IHEM 5452. 10 copies of the DNA target were detected.
(b) Regression analysis of the data used for generation of the standard curve for the ayg1 assay for A. fumigatus. The standard curve shows a PCR efficiency of 1.899 indicating an efficient PCR reaction using the ayg1 target.

### DETAILED DESCRIPTION OF THE DRAWINGS

### Materials and Methods

### Cell Culture

All Aspergillus strains used were grown in sabouraud dextrose broth at 37° C. overnight.

### DNA Extraction

Extraction of DNA from A. fumigatus, Aspergillus species and other organisms, was carried out using the MagNA Pure LC automated nucleic acid extraction system (Roche) using the MagNA Pure LC DNA isolation kit III (Roche) for bacteria and fungi according to manufacturers instructions. DNA was quantified spectrophotometrically at 260/280 nm.

### Sequencing

The publicly available sequence (AF116902, XM 750997) of ayg1 gene in A. fumigatus species available from GenBank was analysed using bioinformatic tools. The PCR primer set Ayg1-F/Ayg1-R (Table 1) was designed to amplify the ayg1 gene region in A. fumigatus. Partial ayg1 sequences for 10 A. fumigatus strains were generated by amplifying the ayg1 gene using primers (ayg1F and ayg1R) designed from the ayg1 gene. Thermocycling was performed on the LightCycler® 1.2 instrument using conditions including a 10-minute denaturation step at 95° C. followed by amplification at 50° C. for 30 seconds and extension at 72° C. for 10 seconds for 45 cycles. All PCR products generated were sequenced. Prior to sequencing, products were treated to remove single stranded DNA and free nucleotides using the PCR product pre-sequencing kit (USB).

Design of a Real-Time PCR Assay for A. Fumigatus Using the ayg1 Gene Target

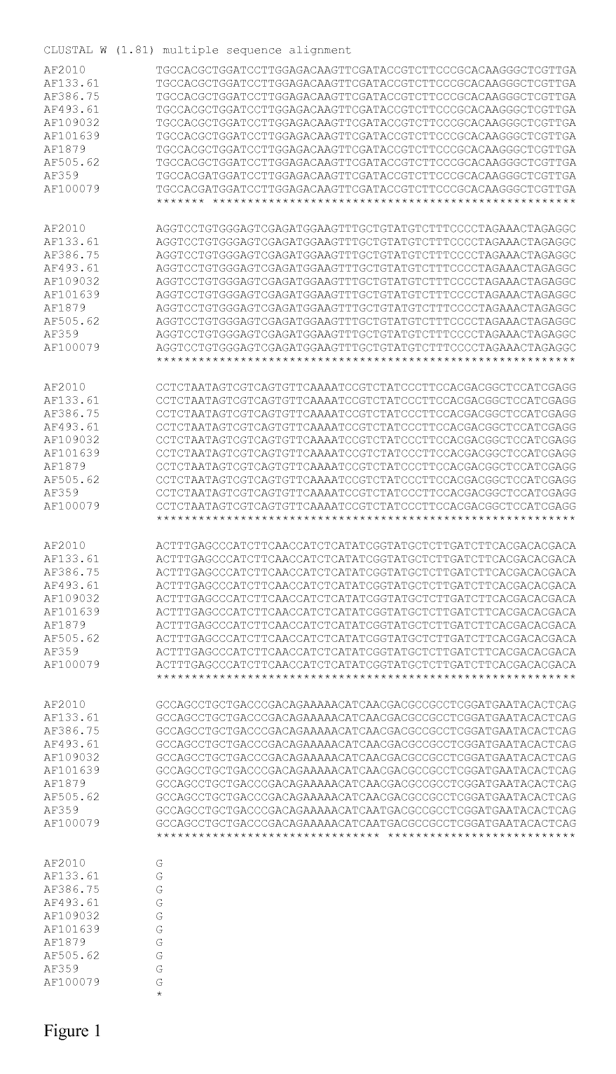

A real time PCR assay for A. fumigatus using the specific gene target ayg1 was developed and demonstrated on LightCycler® real-time PCR instruments. The assay uses two labelled probes (HybProbe) in combination, which operate by the process of fluorescence resonance energy transfer (FRET). The newly generated sequences for the ayg1 gene in A. fumigatus spp. were aligned and analysed using bioinformatics tools. A species-specific HybProbe set (Table 1) was designed based on the compiled ayg1 sequence information for A. fumigatus (FIG. 1).

The HybProbe set was used in combination with the ayg1F and ayg1R forward and reverse primers on the LightCycler®. A HybProbe set works by fluorescence resonance energy transfer (FRET). FRET occurs as the donor fluorophore (fluorescein on the ayg1b probe) is excited photometrically and transfers its energy to the acceptor fluorophore (LC-640 on ayg1b-LC). The acceptor fluorophore emits light at a longer wavelength and is detected by the real-time PCR instrument.

The ayg1 assay for A. fumigatus was performed on the LightCycler® 1.2 and LightCycler® 480 real-time PCR machines (Roche). LightCycler® Faststart DNA Master Hybridization Probes kit (Roche) was used for amplification. On the LightCycler® 1.2 instrument, a final concentration of 4 mM MgCl₂, 0.2 μM hybridisation probe, and 0.5 μM primers were used in addition to 2 μl of template DNA. The thermocycling conditions included a 10 minute denaturation step at 95° C. followed by amplification at 50° C. for 30 seconds and extension at 72° C. for 10 seconds for 45 cycles.

The specificity of a primer and probe combination was verified using DNA extracted from a panel of A. fumigatus reference strains, other Aspergillus species, bacterial species and human DNA.

The detection limit of the assay was determined using serially diluted A. fumigatus DNA (IHEM strain 5452). PCR amplification and detection was performed on the LightCycler® 480 real time PCR machine (Roche). LightCycler® 480 Probes Master kit (Roche) was used for amplification. A final concentration of 6.4 mM MgCl₂, 0.2 μM hybridisation probe, 0.5 μM primers were used in addition to 5 μl of template DNA. The thermocycling conditions included a 10-minute denaturation step at 95° C. followed by amplification at 50° C. for 30 seconds and extension at 72° C. for 10 seconds for 45 cycles.

### Results

### Real-Time PCR

The specificity of the probes for the identification of A. fumigatus was demonstrated by real-time PCR. The PCR primer set Ayg1-F/Ayg1-R was used in combination with the hyb-probe set, Ayg1-LC 640/Ayg1-flu, to detect the ayg1 gene in A. fumigatus. The specificity of the assay for the detection of A. fumigatus was confirmed by including DNA isolated from a range of closely related Aspergillus species, a range of bacterial species and human DNA in the real-time PCR assay (Table 2 and Table 3 and Table 4).

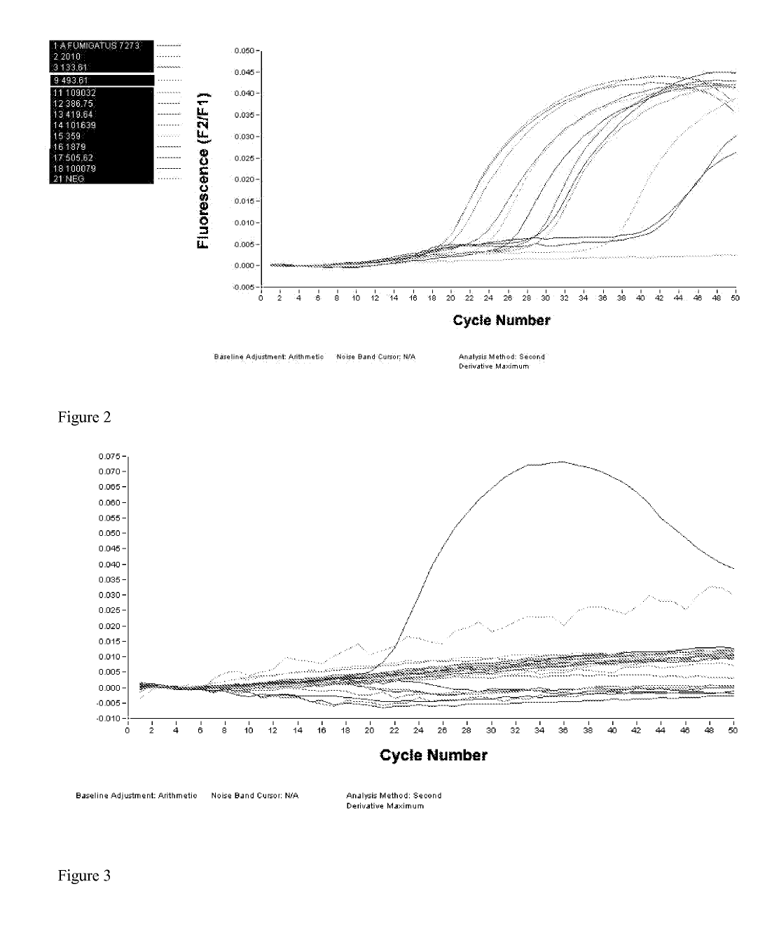

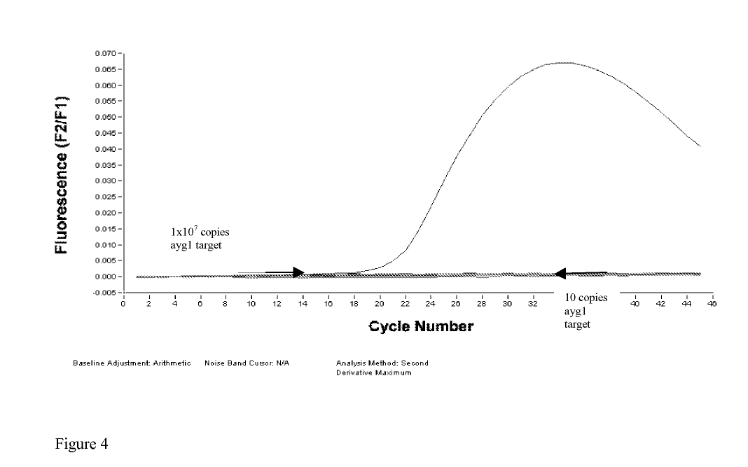

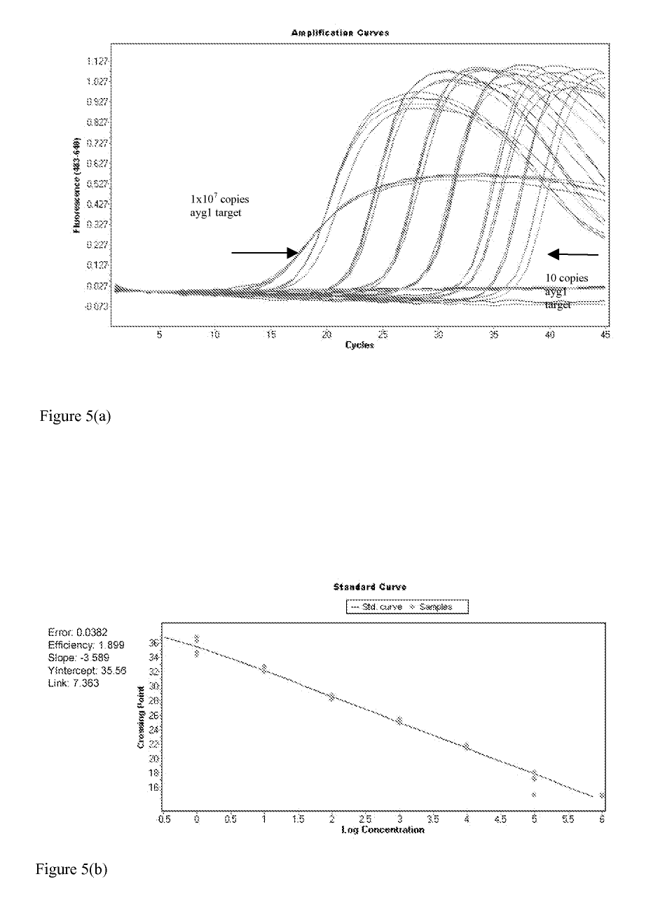

FIG. 2 shows the resulting amplification plot obtained from the real-time PCR assay for all the A. fumigatus strains tested. The assay positively detected the 12 Aspergillus fumigatus strains (n=12) tested. The assay did not detect or cross-react with DNA from other Aspergillus species tested, bacterial species or human DNA, as illustrated in FIGS. 3-4. The detection limit of the assay was established using serially diluted A. fumigatus DNA (IHEM strain 5452) and can reliably detect 10 copies of the gene (FIG. 5a). Therefore, this is not only a specific target it also represents a sensitive target suitable for detection at low copy number. Regression analysis of the data used to generate the standard curve (FIG. 5b) shows a PCR efficiency of 1.899 indicating an efficient PCR reaction.

TABLE 1
Primer andProbe sequences for designed for the
A. fumigatus ayg1 gene

| | |
|---|---|
| Ayg1-F | CATTGCACGTAATCAAGA |
| Ayg1-R | CCTGAGTGTATTCATCCGA |
| Ayg1-b-flu | GACGGCTCCATCGAGGACT |
| Ayg1-b-LC | TGAGCCCATCTTCAACCATCT |

TABLE 2
A. fumigatus strains tested in the ayg1 assay.

| Organism | Strain no | Signal |
|---|---|---|
| Aspergillus fumigatus | NCPF 7273 x 2 | ✓ |
| Aspergillus fumigatus | NCPF 2010 | ✓ |
| Aspergillus fumigatus | CBS 419.64 | ✓ |
| Aspergillus fumigatus | CBS 493.61 | ✓ |
| Aspergillus fumigatus | CBS 100079 | ✓ |
| Aspergillus fumigatus | CBS133.61 | ✓ |
| Aspergillus fumigatus | IHEM 5062 | ✓ |
| Aspergillus fumigatus | CBS 386.75 | ✓ |
| Aspergillus fumigatus | CBS 386.75 | ✓ |
| Aspergillus fumigatus | CBS 109032 | ✓ |
| Aspergillus fumigatus | CBS 101639 | ✓ |
| Aspergillus fumigatus | CBS 419.64 | ✓ |

TABLE 3
Other Aspergillus species tested for cross reactivity

| Organism | Strain no | Signal |
|---|---|---|
| A. nidulans | CBS 670.78 | x |
| A. nidulans | NCPF 2180 | x |
| A. nidulans | CBS 589.65 | x |
| A. versicolor | IHEM 1323 | x |
| A. versicolor | CBS 583.65 | x |
| A. versicolor | IHEM 5058 | x |
| A. flavus | CBS108.30 | x |
| A. flavus | CBS625.66 | x |
| A. flavus | CBS118.62 | x |
| A. terreus | IHEM5918 | x |
| A. terreus | CBS125.35 | x |
| A. terreus | IHEM 5677 | x |
| A. niger | NCPF 2599 | x |
| A. niger | ACBR MA5184 | x |
| A. niger | ACBR MA988 | x |
| A. glaucus | ACBR MA542 | x |
| A. glaucus | ACBR MA5279 | x |
| A. glaucus | IHEM 2425 | x |
| A. clavatus | IHEM 6078 | x |
| A. clavatus | CABI 343709 | x |
| A. candidus | CBS 225.80 | x |
| A. candidus | CBS 102.13 | x |
| A. candidus | CBS 567.65 | x |

TABLE 4
Bacterial species tested in the assay for cross reactivity.

| Organism | Signal |
|---|---|
| E. coli | x |
| P. aeruginosa | x |
| K. pneumoniae | x |
| A. aerogenes | x |
| E. faecium | x |
| S. pneumoniae | x |
| S. maltophilia | x |
| A. baumanii | x |
| K. oxytoca | x |
| P. mirabilis | x |
| E. cloacae | x |
| E. faecalis | x |
| C. freundii | x |
| S. marcescens | x |
| S. aureus | x |

### Discussion

The number of hospital acquired fungal infections occurring worldwide is escalating, resulting in a significant amount of patient deaths each year and placing a significant cost burden on the health sector. The fungal species A. fumigatus is the causative agent of the majority of hospital-acquired infections, including invasive aspergillosis (IA). The increase and severity of such infections, is partly due to the rise in numbers of immunocompromised individuals. Patients particularly at risk include, organ transplant patients, cancer patients and AIDS patients. Further contributing to this increase is the growing resistance of fungal species to antifungal drugs.

The diagnosis of invasive aspergillosis is difficult, particularly in the early stages of disease and at present there is no universally accepted diagnostic method. Difficulties with diagnosis mean that most invasive fungal infections are proven only at autopsy. Current standard laboratory methods lack sensitivity in early detection and are not precise markers of eradication of the infection.

The severity and rapid progression of infection with Aspergillus fumigatus, particularly in immunocomprised patients, illustrate the need for a fast, accurate diagnostic method to enable early diagnosis of IA. Early diagnosis in turn will enable selection of a specific anti-fungal agent to treat the infection.

The current invention provides for sequences and/or diagnostic kit to detect and identify A. fumigatus species. The current inventors have exploited the sequence of the ayg1 gene of A. fumigatus species to design primers and probes specific for regions of this gene.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

In so far as any sequence disclosed herein differs from its counterpart in the attached sequence listing in PatentIn3.3 software, the sequences within this body of text are to be considered as the correct version.

SEQ IDs Sites of probes, oligonucleotides etc. are shown in bold and underlined. N or x=any nucleotide; w=a/t, m=a/c, r=a/g, k=g/t, s=c/g, y=c/t, h=a/t/c, v=a/g/c, d=a/g/t, b=g/t/c. In some cases specific degeneracy options are indicated in parenthesis e.g. (a/g) is either A or G.

SEQ ID NO. 1: ayg1F:
5′ CACTGCACGTAATCAAGA 3′
SEQ ID NO. 2: ayg1R:
5′CCTGAGTGTATTCATCCGA 3′
SEQ ID NO. 3: ayg1-LC 640
5′ TGAGCCCATCTTCAACCATCT 3′
SEQ ID NO. 4: ayg1-flu
5′GACGGCTCCATCGAGGACT 3′
SEQ ID NO. 5:
>AF2010
TGCCACGCTGGATCCTTGGAGACAAGTTCGATACCGTCTTCCCGCACAAGGGCTCG
TTGAAGGTCCTGTGGGAGTCGAGATGGAAGTTTGCTGTATGTCTTTCCCCTAGAAAC
TAGAGGCCCTCTAATAGTCGTCAGTGTTCAAAATCCGTCTATCCCTTCCACGACGGC
TCCATCGAGGACTTTGAGCCCATCTTCAACCATCTCATATCGGTATGCTCTTGATCT
TCACGACACGACAGCCAGCCTGCTGACCCGACAGAAAAACATCAACGACGCCGCCT
CGGATGAATACACTCAGG
SEQ ID NO 6
>AF133.61
TGCCACGCTGGATCCTTGGAGACAAGTTCGATACCGTCTTCCCGCACAAGGGCTCG
TTGAAGGTCCTGTGGGAGTCGAGATGGAAGTTTGCTGTATGTCTTTCCCCTAGAAAC
TAGAGGCCCTCTAATAGTCGTCAGTGTTCAAAATCCGTCTATCCCTTCCACGACGGC
TCCATCGAGGACTTTGAGCCCATCTTCAACCATCTCATATCGGTATGCTCTTGATCT
TCACGACACGACAGCCAGCCTGCTGACCCGACAGAAAAACATCAACGACGCCGCCT
CGGATGAATACACTCAGG
SEQ ID NO 7
>AF493.61
TGCCACGCTGGATCCTTGGAGACAAGTTCGATACCGTCTTCCCGCACAAGGGCTCG
TTGAAGGTCCTGTGGGAGTCGAGATGGAAGTTTGCTGTATGTCTTTCCCCTAGAAAC
TAGAGGCCCTCTAATAGTCGTCAGTGTTCAAAATCCGTCTATCCCTTCCACGACGGC
TCCATCGAGGACTTTGAGCCCATCTTCAACCATCTCATATCGGTATGCTCTTGATCT
TCACGACACGACAGCCAGCCTGCTGACCCGACAGAAAAACATCAACGACGCCGCCT
CGGATGAATACACTCAGG
SEQ ID NO 8
>AF109032
TGCCACGCTGGATCCTTGGAGACAAGTTCGATACCGTCTTCCCGCACAAGGGCTCG
TTGAAGGTCCTGTGGGAGTCGAGATGGAAGTTTGCTGTATGTCTTTCCCCTAGAAAC
TAGAGGCCCTCTAATAGTCGTCAGTGTTCAAAATCCGTCTATCCCTTCCACGACGGC
TCCATCGAGGACTTTGAGCCCATCTTCAACCATCTCATATCGGTATGCTCTTGATCT
TCACGACACGACAGCCAGCCTGCTGACCCGACAGAAAAACATCAACGACGCCGCCT
CGGATGAATACACTCAGG
SEQ ID NO 9
>AF386.75
TGCCACGCTGGATCCTTGGAGACAAGTTCGATACCGTCTTCCCGCACAAGGGCTCG
TTGAAGGTCCTGTGGGAGTCGAGATGGAAGTTTGCTGTATGTCTTTCCCCTAGAAAC
TAGAGGCCCTCTAATAGTCGTCAGTGTTCAAAATCCGTCTATCCCTTCCACGACGGC
TCCATCGAGGACTTTGAGCCCATCTTCAACCATCTCATATCGGTATGCTCTTGATCT
TCACGACACGACAGCCAGCCTGCTGACCCGACAGAAAAACATCAACGACGCCGCCT
CGGATGAATACACTCAGG
SEQ ID NO 10
>AF101639
TGCCACGCTGGATCCTTGGAGACAAGTTCGATACCGTCTTCCCGCACAAGGGCTCG
TTGAAGGTCCTGTGGGAGTCGAGATGGAAGTTTGCTGTATGTCTTTCCCCTAGAAAC
TAGAGGCCCTCTAATAGTCGTCAGTGTTCAAAATCCGTCTATCCCTTCCACGACGGC
TCCATCGAGGACTTTGAGCCCATCTTCAACCATCTCATATCGGTATGCTCTTGATCT
TCACGACACGACAGCCAGCCTGCTGACCCGACAGAAAAACATCAACGACGCCGCCT
CGGATGAATACACTCAGG
SEQ ID NO 11
>AF359
TGCCACGATGGATCCTTGGAGACAAGTTCGATACCGTCTTCCCGCACAAGGGCTCG
TTGAAGGTCCTGTGGGAGTCGAGATGGAAGTTTGCTGTATGTCTTTCCCCTAGAAAC
TAGAGGCCCTCTAATAGTCGTCAGTGTTCAAAATCCGTCTATCCCTTCCACGACGGC
TCCATCGAGGACTTTGAGCCCATCTTCAACCATCTCATATCGGTATGCTCTTGATCT
TCACGACACGACAGCCAGCCTGCTGACCCGACAGAAAAACATCAATGACGCCGCCT
CGGATGAATACACTCAGG
SEQ ID NO 12
>AF1879
TGCCACGCTGGATCCTTGGAGACAAGTTCGATACCGTCTTCCCGCACAAGGGCTCG
TTGAAGGTCCTGTGGGAGTCGAGATGGAAGTTTGCTGTATGTCTTTCCCCTAGAAAC
TAGAGGCCCTCTAATAGTCGTCAGTGTTCAAAATCCGTCTATCCCTTCCACGACGGC
TCCATCGAGGACTTTGAGCCCATCTTCAACCATCTCATATCGGTATGCTCTTGATCT
TCACGACACGACAGCCAGCCTGCTGACCCGACAGAAAAACATCAACGACGCCGCCT
CGGATGAATACACTCAGG
SEQ ID NO 13
>AF505.62
TGCCACGCTGGATCCTTGGAGACAAGTTCGATACCGTCTTCCCGCACAAGGGCTCG
TTGAAGGTCCTGTGGGAGTCGAGATGGAAGTTTGCTGTATGTCTTTCCCCTAGAAAC
TAGAGGCCCTCTAATAGTCGTCAGTGTTCAAAATCCGTCTATCCCTTCCACGACGGC
TCCATCGAGGACTTTGAGCCCATCTTCAACCATCTCATATCGGTATGCTCTTGATCT
TCACGACACGACAGCCAGCCTGCTGACCCGACAGAAAAACATCAACGACGCCGCCT
CGGATGAATACACTCAGG
SEQ ID NO 14
>AF100079
TGCCACGATGGATCCTTGGAGACAAGTTCGATACCGTCTTCCCGCACAAGGGCTCG
TTGAAGGTCCTGTGGGAGTCGAGATGGAAGTTTGCTGTATGTCTTTCCCCTAGAAAC
TAGAGGCCCTCTAATAGTCGTCAGTGTTCAAAATCCGTCTATCCCTTCCACGACGGC
TCCATCGAGGACTTTGAGCCCATCTTCAACCATCTCATATCGGTATGCTCTTGATCT
TCACGACACGACAGCCAGCCTGCTGACCCGACAGAAAAACATCAATGACGCCGCCT
CGGATGAATACACTCAGG

### REFERENCES

1. Balajee, S. A., Houbraken, J., Verweij, P. E., Hong, S-B., Yaghuchi, T., Varga, J. and Samson, R. A. (2007). Aspergillus species identification in the clinical setting. Studies in Mycology, 59: 39-46.
2. Speiss, B., Seifarth, W., Hummel, M., Frank, O, Fabarius, A., Zheng, C., Morz, H., Hehlmann, R. and Buchheidt, DD. (2007). DNA microarray-based detection and identification of fungal pathogens in clinical samples from neutropenic patients. J. Clin. Microbiol. 45, 3743-3753.
3. Lau, A., Chen, S., Sorrell, T., Carter, D., Malik, R., Martin, P. and Halliday, C. (2007). Development and clinical application of a panfungal PCR assay to detect and identify fungal DNA in tissue specimens. J. Clin. Microbiol. 45, 380-385.
4. DeMarco, D., Perotti, M., Ossi, C. M., Burioni, R., Clementi, M. and Mancini, N. (2007). Development and validation of a molecular method for the diagnosis of medically important fungal infections. New Microbiol. 30: 308-312.
5. HUEI-FUNG TSAI, WHEELER, M. H., CHANG, YUN C. and KWON-CHUNG, K. J. (1999) A Developmentally Regulated Gene Cluster Involved in Conidial Pigment Biosynthesis in Aspergillus fumigatus. JOURNAL OF BACTERIOLOGY, 6469-6477 Vol. 181, No. 20

1. A diagnostic kit for Aspergillus fumigatus comprising at least one oligonucleotide probe capable of hybridizing to at least a portion of the ayg1 gene or its corresponding mRNA.

2. The kit as claimed in claim 1 wherein the portion of the ayg1 gene is a portion of base pair position 316 to base pair 634 of the ayg1 gene.

3. The kit as claimed in claim 1, wherein the probe is selected from the group consisting of SEQ ID NO 3, SEQ ID NO 4, and sequences substantially similar or complementary thereto.

4. The kit as claimed in claim 1, further comprising at least one primer for amplification of at least a portion of the ayg1 gene.

5. The kit as claimed in claim 4, comprising a forward and/or a reverse primer for a portion of the ayg1 gene.

6. The kit as claimed in claim 4 comprising at least one forward in vitro amplification primer and/or at least one reverse in vitro amplification primer, wherein the forward amplification primer is SEQ ID NO 1, or sequences substantially similar or complementary thereto, and wherein the reverse amplification primer is SEQ ID NO 2 or sequences substantially similar or complementary thereto.

7. An oligonucleotide sequence selected from the group consisting of: SEQ ID NO 1 through SEQ ID NO 14, sequences substantially homologous or substantially complementary thereto, and sequences substantially homologous or substantially complementary to a portion thereof.

8. A method of detecting Aspergillus fumigatus comprising the step of using the oligonucleotide sequence as claimed in claim 7 in a diagnostic kit.

9. A method of detecting a target organism in a test sample comprising the steps of:
(i) Mixing the test sample with at least one oligonucleotide sequence capable of binding to at least a portion of the ayg1 gene or its corresponding mRNA;
(ii) Hybridizing nucleic acid in the test sample with the oligonucleotide sequence; and
(iii) Determining whether a sequence:target duplex is present.

10. The method as claimed in claim 9 wherein the sequence is selected from the group consisting of SEQ ID No 1 to SEQ ID No 4, and sequences substantially homologous or substantially complementary thereto.

11. The method as claimed in claim 9, wherein the sample is an environmental sample, an industrial sample, a clinical sample or a food sample.

12-22. (canceled)

23. A method of measuring fungal titers in a patient comprising the step of using the kit of claim 1 in a diagnostic assay.

24. A method of assessing the efficacy and response of a treatment regime of a patient comprising the step of using the kit of claim 1.

25. A method of measuring fungal contamination in a sample comprising the step of using the kit of claim 1 in a diagnostic assay.

26. The method of claim 25, wherein the sample is a sample in a hospital, a food sample, an environmental sample or a clinical sample.

27. A method for identifying or characterizing at least one disruptive agent that can be used to disrupt the ayg1 gene function comprising the step of using the kit of claim 1.

28. The method of claim 27, wherein the disruptive agent is selected from the group consisting of antisense RNA, PNA, siRNA and miRNA.

29. A method for assessing the efficacy of a treatment regime designed to reduce fungal titer in a patient comprising the step of using the kit of claim 1.

30. A method of measuring fungal titers in a patient comprising the step of using the oligonucleotide sequence of claim 7 in a diagnostic assay.

31. A method of assessing the efficacy and response of a treatment regime of a patient comprising the step of using the oligonucleotide sequence of claim 7.

32. A method of measuring fungal contamination in a sample comprising the step of using the oligonucleotide sequence of claim 7 in a diagnostic assay.

33. The method of claim 32, wherein the sample is a sample in a hospital, a food sample, an environmental sample or a clinical sample.

34. A method for identifying or characterizing at least one disruptive agent that can be used to disrupt the ayg1 gene function comprising the step of using the oligonucleotide sequence of claim 7.

35. The method of claim 34, wherein the disruptive agent is selected from a group consisting of antisense RNA, PNA, siRNA and miRNA.

36. A method for assessing the efficacy of a treatment regime designed to reduce fungal titer in a patient comprising the step of using the oligonucleotide sequence of claim 7.

37. The method of claim 9, further comprising the step of measuring fungal titers in a patient, wherein the target organism is a fungus, and wherein the test sample is from the patient.

38. The method of claim 9, further comprising the step of assessing the efficacy and response of a treatment regime of a patient, wherein the test sample is from the patient.

39. The method of claim 9, further comprising the step of measuring fungal contamination in the test sample, wherein the target organism is a fungus.

40. The method of claim 39, wherein the test sample is a sample in a hospital, a food sample, an environmental sample or a clinical sample.

41. The method of claim 9, further comprising the step of identifying or characterizing at least one disruptive agent that can be used to disrupt the ayg1 gene function.

42. The method of claim 41, wherein the disruptive agent is selected from the group consisting of antisense RNA, PNA, siRNA and miRNA.

43. The method of claim 9, further comprising the step of assessing the efficacy of a treatment regime designed to reduce fungal titer in a patient, wherein the target organism is a fungus, and wherein the test sample is from the patient.
